(19) United States
(12) Patent Application Publication
(10) Pub. No.: US 2026/0274791 A1
(43) Pub. Date: Sep. 17, 2026

(54) ADDITIVE FOR ORGANIC CONDUCTORS

(71) Applicant: Panasonic Intellectual Property Management Co., Ltd., Osaka (JP)

(72) Inventors: Keisuke HAYASHI, OSAKA FU (JP); Nobuyuki MATSUZAWA, TOKYO TO (JP); Toshiyuki TAKIZAWA, KYOTO FU (JP); Hiroyuki MAESHIMA, HYOGO KEN (JP)

(21) Appl. No.: 18/871,746

(22) PCT Filed: Aug. 30, 2023
(86) PCT No.: PCT/JP2023/031456

§ 371 Date: Dec. 4, 2024

(30) Foreign Application Priority Data
Sep. 15, 2022 (JP) 2022-147410

(51) Int. Cl. C07C 309/58; H01G 9/025; H01G 9/048; H01G 9/15

(52) U.S. Cl. CPC C07C 309/58; H01G 9/025; H01G 9/048; H01G 9/15

(57) ABSTRACT

An additive for an organic conductor has a naphthalene ring, a sulfo group bonded to the naphthalene ring, and a carboxy group bonded to the naphthalene ring. Where the carbon atom of the naphthalene ring to which the sulfo group is bonded and the carbon atom of the naphthalene ring to which the carboxy group is bonded are a first carbon atom and a second carbon atom, respectively, the number n of carbon atoms between the first carbon atom and the second carbon atom is 3 or less. This makes it possible to provide an additive that can remarkably increase moisture resistance of the organic conductor.

### TECHNICAL FIELD

The present disclosure relates to an additive for organic conductors, as well as an organic conductor and an electrolytic capacitor that use the same.

### BACKGROUND ART

Conjugated polymers such as polythiophene and polypyrrole show conductivity with addition of a dopant. Conjugated polymers to which a dopant is added are called conductive polymers or organic conductors. In recent years, an organic conductor of self-doping type has also been developed. Organic conductors are used in various electronic components because they are inexpensive and lightweight and their performance can be controlled by selecting, for example, the type of conjugated polymer or the type of additive (e.g., a dopant). As the dopant being an additive, a proton-added compound or an electronic-oxidized compound is used, for example.

For example, it is proposed to add an organic sulfonic acid to a solid electrolyte layer of a solid electrolytic capacitor (Patent Literatures 1 and 2)

### CITATION LIST

### Patent Literature

Patent Literature 1: Japanese Laid-Open Patent Publication No. 2006-108650
Patent Document 2: International Publication No. WO 2019/131476

### SUMMARY OF INVENTION

### Technical Problem

When an electrolytic capacitor with an organic conductor is operated in a high-humidity environment, capacitance degradation and an increase in equivalent series resistance (ESR) are caused.

### Solution to Problem

A first aspect of the present disclosure relates to an additive for an organic conductor, including a naphthalene compound having a naphthalene ring, a sulfo group bonded to the naphthalene ring, and a carboxy group bonded to the naphthalene ring, wherein a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded, and the naphthalene compound has a purity of 41 mass % or more.

A second aspect of the present disclosure relates to an organic conductor including a conjugated polymer and the above-described additive.

A third aspect of the present disclosure relates to an electrolytic capacitor including: an anode body including a dielectric layer on a surface thereof; and a solid electrolyte covering a portion of the dielectric layer, wherein the solid electrolyte contains the above-described organic conductor.

A fourth aspect of the disclosure relates to an electrolytic capacitor including: an anode body including a dielectric layer on a surface thereof; and a solid electrolyte covering a portion of the dielectric layer, wherein the solid electrolyte contains an organic conductor, the organic conductor contains a conjugated polymer and a dopant, a naphthalene compound constitutes 80 mass % or more of the dopant, the naphthalene compound has a naphthalene ring, a sulfo group bonded to the naphthalene ring, and a carboxy group bonded to the naphthalene ring, a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded.

A fifth aspect of the present disclosure relates to an additive for an organic conductor, wherein a content ratio of at least one selected from the group consisting of a 7-sulfo-2-naphthoic acid compound, a 7-sulfo-1-naphthoic acid compound, and a 6-sulfo-1-naphthoic acid compound is 80 mass % or more.

A sixth aspect of the present disclosure relates to an additive for an organic conductor, wherein a content ratio of at least one selected from the group consisting of 7-sulfo-2-naphthoic acid and a 7-sulfo-1-naphthoic acid compound is 80 mass % or more.

A seventh aspect of the present disclosure relates to an organic conductor including: a conjugated polymer; and a dopant, wherein 80 mass % or more of the dopant is at least one selected from the group consisting of a 7-sulfo-2-naphthoic acid compound, a 7-sulfo-1-naphthoic acid compound, and a 6-sulfo-1-naphthoic acid compound.

An eighth aspect of the present disclosure relates to an organic conductor including: a conjugated polymer; and a dopant, wherein 80 mass % or more of the dopant is at least one selected from the group consisting of 7-sulfo-2-naphthoic acid and a 7-sulfo-1-naphthoic acid compound.

### Advantageous Effects of Invention

It is possible to provide an additive that can significantly increase moisture resistance of an organic conductor, an organic conductor having excellent moisture resistance, and an electrolytic capacitor using the organic conductor.

While the novel features of the invention are set forth in the appended claims, the invention, both as to organization and content, will be better understood and appreciated, along with other objects and features thereof, from the following detailed description taken in conjunction with the drawings.

### BRIEF DESCRIPTION OF THE DRAWINGS

FIG. 1 is a schematic cross-sectional view of an electrolytic capacitor according to an embodiment of the present disclosure.

### DESCRIPTION OF EMBODIMENTS

Embodiments of the present disclosure are described below by way of examples, but the present disclosure is not limited to the examples described below. In the following description, specific numerical values and materials may be exemplified in some cases, but other numerical values and other materials may be adopted as long as the effects of the present disclosure can be obtained. In the present description, the phrase “a numerical value A to a numerical value B” means to include the numerical value A and the numerical value B, and can be phrased as “a numerical value A or more and a numerical value B or less”. In the following description, when the lower and upper limits of numerical values related to specific physical properties, conditions, or the like are mentioned as examples, any of the mentioned lower limits and any of the mentioned upper limits can be combined in any combination as long as the lower limit is not equal to or more than the upper limit.

The present disclosure encompasses a combination of matters recited in any two or more claims selected from multiple claims in the appended claims. In other words, as long as no technical contradiction arises, matters recited in any two or more claims selected from multiple claims in the appended claims can be combined.

In the following description, the word “comprise” or “include” is an expression including meanings of “comprise (or include)”, “essentially consist of”, and “consist of”.

An additive for an organic conductor of the present disclosure includes a naphthalene compound (also referred to below as “naphthalene compound D”) having a naphthalene ring, a sulfo group bonded to the naphthalene ring, and a carboxy group bonded to the naphthalene ring. A first carbon atom and a second carbon atom are adjacent to each other, or the number n of carbon atoms between the first carbon atom and the second carbon atom is 3 or less. Here, the first carbon atom is a carbon atom of the naphthalene ring to which the sulfo group is bonded, and the second carbon atom is a carbon atom of the naphthalene ring to which the carboxy group is bonded.

The naphthalene compound D has an action of extracting electrons of a conjugated polymer in order to convert an insulator or a semiconductor into a good conductor. Therefore, an additive having such an action is generally called a dopant. In the present description, an additive or the naphthalene compound D can also be referred to as a “dopant”.

It was revealed that when an organic conductor containing a conjugated polymer and the naphthalene compound D having a purity of 41 mass % or more is used for a solid electrolyte of an electrolytic capacitor, the rate of change of ESR during operation of the electrolytic capacitor in a high-humidity environment can be significantly reduced. The naphthalene compound D may have a purity of 45 mass % or more, 50 mass % or more, 60 mass % or more, 70 mass % or more, or 80 mass % or more.

The purity of the naphthalene compound D being 41 mass % or more means that the content ratio of one type of naphthalene compound included in the category of the naphthalene compound D is 41 mass % or more and at least any of another naphthalene compound D included in the category of the naphthalene compound D, a naphthalene compound not included in the category of the naphthalene compound D, and an impurity that is not a naphthalene compound is present at less than 59 mass %.

In general, operation of an electrolytic capacitor with an organic conductor in a high-humidity environment causes capacity degradation and the ESR increases. This is because water molecules are adsorbed in the organic conductor, which can peel the additive off the conjugated polymer.

The rate of change of ESR during operation in a high-humidity environment may be also referred to simply as ΔESR. The ΔESR particularly tends to increase when the electrolytic capacitor is operated at high temperatures (e.g., 80° C. or more). Therefore, even if the increase in ΔESR at relatively low temperatures (e.g., 60° C. or less) does not pose a significant problem, the increase in ΔESR at high temperatures becomes significant.

The naphthalene compound D has at least one sulfo group and one carboxy group at positions relatively close to each other in the naphthalene ring. This makes the at least one sulfo group and one carboxy group more accessible to the conjugated polymer. Therefore, the bonding strength between the naphthalene compound D and the conjugated polymer is thought to increase. In other words, even if the organic conductor adsorbs water molecules in a high-humidity environment, peeling of the molecules of the naphthalene compound D from the conjugated polymer is significantly inhibited to suppress an increase in resistance of the organic conductor, thereby maintaining high conductivity. Thus, when an organic conductor containing a high-purity naphthalene compound D as a dopant is used for a solid electrolyte of an electrolytic capacitor, the A ESR can be significantly reduced. Such an advantage can be ensured even in high-temperature (e.g., 80° C. or more) and high-humidity environments.

The position numbers of the carbons constituting the naphthalene ring are as shown below in Formula (I).

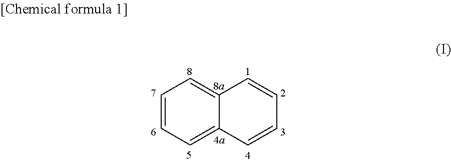

In the present description, the number n is the number of carbon atoms located between the first carbon atom and the second carbon atom when attention is paid to the shortest carbon chain among the carbon chains connecting the first carbon atom and the second carbon atom in the naphthalene ring. As such, the number n of the carbon atoms located between the first carbon atom and the second carbon atom is determined to be the smallest. The number n does not include the numbers of the first carbon atom and the second carbon atom. When the first carbon atom and the second carbon atom are adjacent to each other, the number n is 0

Note that the carbon chain connecting the first carbon atom and the second carbon atom is a carbon chain constituting the naphthalene ring, and does not include a substituent that the naphthalene ring has. For example, in a case of 7-sulfo-2-naphthoic acid represented by the following formula (ia), the shortest carbon chain is a carbon chain where the carbon atoms at positions 1, 8a and 8 are linked in the stated order among the carbon chains connecting the first carbon atom at position 7 and the second carbon atom at position 2. There are three carbon atoms between the first carbon atom at position 7 and the second carbon atom at position 2. The number n is accordingly 3. In a case of 7-sulfo-1-naphthoic acid represented by the following formula (ib), the shortest carbon chain is a carbon chain where the carbon atoms at 8a position and 8 position are linked in the stated order among the carbon chains connecting the first carbon atom at position 7 and the second carbon atom at position 1. There are two carbon atoms located at 8a and 8 positions between the first carbon atom at 7 position and the second carbon atom at position 1. The number n is accordingly 2.

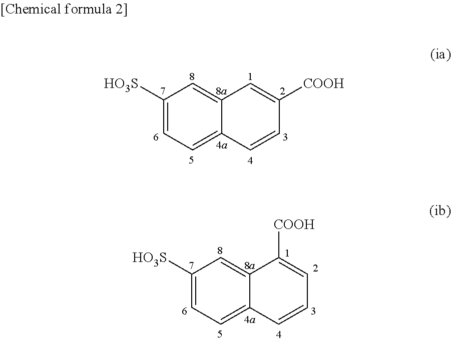 [SMILES: O=C(O)c1ccc2ccc(S(=O)(=O)O)cc2c1 ; O=C(O)c1cccc2ccc(S(=O)(=O)O)cc12]

For example, when each number of the sulfo groups and the carboxy groups is 1, n is 3 or less. Therefore, the naphthalene compound D does not encompasses a compound in which the sulfo group and the carboxy group are respectively bonded to positions 2 and 6 of the naphthalene ring, a compound in which the sulfo group and the carboxy group are respectively bonded to positions 3 and 7, a compound in which the sulfo group and the carboxy group are respectively bonded to positions 1 and 5, and a compound in which the sulfo group and the carboxy group are respectively bonded to positions 4 and 8. This is because n becomes 4 in these compounds.

The sulfo group may be included in the naphthalene compound D or the organic conductor in the form of a free (—SO₃H) or an anion (—SO₃—), or may be included in the form of a salt. In the organic conductor, the sulfo group may be included in the form of being bonded to the conjugated polymer or interacting with the conjugated polymer. In the present description, sulfo groups including groups in any of these forms may be referred to simply as “sulfo groups.” Likewise, the carboxy group may be included in the naphthalene compound D or the organic conductor in the form of a free (—COOH) or an anion (—COO—), or may be included in the form of a salt. In the present description, carboxy groups including groups in any of these forms may be referred to simply as “carboxy groups.” Note that a salt may be a salt of a sulfonic anion or a carboxylate anion and any of an organic base (such as an organic amine or an organic ammonium), an inorganic base (such as a metal hydroxide or ammonia), and a metal cation (such as Na or Li).

The following describes the additive (naphthalene compound D) for organic conductors of the present disclosure, a production method thereof, an organic conductor, and an electrolytic capacitor in detail.

### [Additive for Organic Conductors]

When the naphthalene compound D has one sulfo group and one carboxy group, the number n of carbon atoms present between the first carbon atom and the second carbon atom to which respective substituents are bonded is 0 to 3. One sulfo group and one carboxy group are located relatively close to each other in the naphthalene ring of the naphthalene compound D. Thus, as described above, it is thought that a high bonding strength to the conjugated polymer can be obtained and an increase in resistance of an organic conductor can be suppressed even in high-humidity environments.

The number of the sulfo groups is at least 1, and may be 1 to 5, or may be 1 or 2. From the point of view where electron-withdrawing ability of the carboxy group is easily exerted, the number of the sulfo groups is preferably 1.

The number of the carboxy groups is at least 1, and may be 1 to 6 or 1 to 4, or may be 1 or 2. However, when the number of the carboxy groups in the naphthalene ring is 3 or more, steric repulsion for the conjugated polymer may increase. Therefore, the number of the carboxy groups is preferably 2 or less, and more preferably 1 rather than 2.

The naphthalene compound D may have a first substituent other than the sulfo group and the carboxy group in the naphthalene ring. Compounds with the first substituent are also encompassed in the additive of the present disclosure. The first substituent may be, for example, an electron-donating group or an electron-withdrawing group other than the sulfo group and the carboxy group. However, a hydrocarbon group is preferable because of its ability to easily exhibit a higher electron acceptor function, which is based on the balance between the sulfo group and the carboxy group.

The hydrocarbon group may be any of the following: fatty, alicyclic, and aromatic. From the point of view of easy coordination to the conjugated polymer, the hydrocarbon group is preferably an aliphatic hydrocarbon group. The number of carbon atoms of the aliphatic hydrocarbon group is, for example, 1 to 10, and may be 1 to 6 or 1 to 4. The aliphatic hydrocarbon group may be either saturated or unsaturated.

Examples of the aliphatic hydrocarbon group include an alkyl group, an alkenyl group, an alkynyl group, and a dienyl group. Among these groups, an alkyl group is preferable. Examples of the alkyl group include a methyl group, an ethyl group, an n-propyl group, an isopropyl group, a n-butyl group, an isobutyl group, a sec-butyl group, a tert-butyl group, a hexyl group, an octyl group, and a 2-ethylhexyl group.

The naphthalene compound D may have one first substituent or may have two or more first substituents. When the naphthalene compound D has two or more first substituents, at least two of the first substituents may be the same or all of the first substituents may be different.

From the point of view of easy exhibition of a higher electron acceptor function based on the balance between the sulfo group and the carboxy group, it is also preferable that the naphthalene compound D does not have the first substituent.

A non-aromatic ring Z may be fused to the naphthalene ring of the naphthalene compound D. In the naphthalene compound D having such a structure, for example, a plurality of (e.g., 2) carbon atoms among the carbon atoms at positions 1 to 8 of the naphthalene ring are connected by an aliphatic chain. The aliphatic chain may be saturated or unsaturated. One example of such a structure is an acenaphthene ring.

The naphthalene compound D may have 1 or 2 or more second substituents in the aliphatic chain. Examples of the second substituents include a sulfo group, a carboxy group, and the groups described for the first substituent. When the naphthalene compound D has 2 or more second substituents, at least 2 of the second substituents may be the same or all of the second substituents may be different.

From the point of view of easy access to the conjugated polymer, it is preferable that the naphthalene ring of the naphthalene compound D is not fused with a non-aromatic ring Z, such as described above.

From the point of view of enhancing the effect of suppressing an increase in resistance of the organic conductor in high-humidity environments, the naphthalene compound D preferably has one sulfo group and one carboxy group. Above all, it is preferable that one group selected from these 2 groups is bonded to one of the benzene rings constituting the naphthalene ring, and the other 1 group is bonded to the other benzene ring.

From the point of view of further suppressing an increase in resistance of the organic conductor in high-humidity environments, the number of carbon atoms between the sulfo group and the carboxy group is preferably 2 or less. When the number of carbon atoms between the sulfo group and the carboxy group is 2 or less, both of the sulfo group and the carboxy group become more accessible to the conjugated polymer, so that it is thought that conductivity of the organic conductor can be easily increased.

As a result of the naphthalene compound D having the structure as described above, a high bonding strength to the conjugated polymer can be ensured. The additive has an interaction energy to the conjugated polymer of preferably −13 kcal/mol or less, more preferably −14 kcal/mol or less, further preferably −15 kcal/mol or less, and may be a low value of −17 kcal/mol or less or −19 kcal/mol or less. The interaction energy of naphthalene sulfonic acid, which is generally used as a dopant, and polypyrrole is about −10 kcal/mol.

The interaction energy of the naphthalene compound D to the conjugated polymer is obtained by subtracting the respective potential energies of the naphthalene compound D and the conjugated polymer when each is present alone from the potential energy of a complex of the naphthalene compound D and the conjugated polymer. Each potential energy can be obtained from the Schrödinger equation using quantum chemical computational software (Gaussian Inc., Gaussian 09).

The naphthalene compound D can be represented also by the following formula (1).

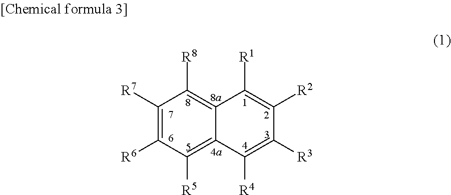

where in the formula, R¹ to R⁸ each represent a hydrogen atom, a sulfo group, a carboxy group, or the first substituent, at least one of R¹ to R⁸ is a sulfo group, at least one of R¹ to R⁸ is a carboxy group, and 2 groups selected from R¹ to R⁸ may be bonded to each other to form anon-aromatic ring Z fused to the naphthalene ring. The ring Z may have a second substituent. When two groups of R¹ to R⁸ each represent a sulfo group, cases in which each of R² and R⁶ represents a sulfo group, and R³ and R⁷ each represent a sulfo group are excluded.

In formula (1), the above description can be referenced for the sulfo group, the carboxy group, and their numbers and locations. For the first substituent, the ring Z, and the second substituent, the above description can be also referenced.

The naphthalene compound D is preferably at least one selected from the group consisting of a 7-sulfo-2-naphthoic acid compound, a 7-sulfo-1-naphthoic acid compound, a 6-sulfo-1-naphthoic acid compound, a 1-sulfo-2-naphthoic acid compound, and a 4-sulfo-1-naphthoic acid compound. More preferable one is at least one selected from the group consisting of a 7-sulfo-2-naphthoic acid compound, a 7-sulfo-1-naphthoic acid compound, and a 6-sulfo-1-naphthoic acid compound. Each compound includes, for example, sulfonaphthoic acid having a first substituent besides sulfonaphthoic acid. Above all, 7-sulfo-2-naphthoic acid or a 7-sulfo-1-naphthoic acid compound is preferable.

The content ratio of at least one selected from the group consisting of a 7-sulfo-2-naphthoic acid compound, a 7-sulfo-1-naphthoic acid compound, and a 6-sulfo-1-naphthoic acid compound in the additive for an organic conductor may be 80 mass % or more, for example. The content ratio of at least one selected from the group consisting of 7-sulfo-2-naphthoic acid and a 7-sulfo-1-naphthoic acid compound in the additive for an organic conductor may be 80 mass % or more, for example.

One type of the naphthalene compound D may be used singly, or two or more types thereof may be used in combination.

The additive preferably includes 80% or more and 100% or less of the naphthalene compound D in terms of mass, more preferably 90% or more and 100% or less, and further more preferably 95% or more and 100% or less.

### [Organic Conductor]

The organic conductor includes a conjugated polymer and a dopant including a naphthalene compound D having a high purity of 41 mass % or more. Preferably, 80 mass % or more of the dopant contained in the organic conductor is the naphthalene compound D. At least one selected from the group consisting of a 7-sulfo-2-naphthoic acid compound, a 7-sulfo-1-naphthoic acid compound, and a 6-sulfo-1-naphthoic acid compound may constitute 80 mass % or more of the dopant. At least one selected from the group consisting of 7-sulfo-2-naphthoic acid and a 7-sulfo-1-naphthoic acid compound may constitute 80 mass % or more of the dopant. The naphthalene compound D exhibits excellent electron acceptor performance and can effectively function as a dopant to the conjugated polymer. Use of the naphthalene compound D can achieve a high bonding strength to the conjugated polymer. Therefore, even if the organic conductor adsorbs water molecules in a high-humidity environment, dedoping can be suppressed to suppress an increase in Δ ESR. Thus, high reliability of electrolytic capacitors can be ensured even when used in high-humidity environments.

The conjugated polymer should be any polymer that becomes a good conductor through the action of a dopant, and examples thereof include a π-conjugated polymer and a σ-conjugated polymer. The organic conductor may contain one type of naphthalene compound D as a dopant, or may contain 2 or more types thereof. The organic conductor may contain one type of conjugated polymer, or may include 2 or more types thereof.

Examples of the conjugated polymer include polymers with a basic skeleton of polymers such as polypyrrole, polythiophene, polyaniline, polyfuran, polyacetylene, polyphenylene, polyphenylene vinylene, polyacene, and polythiophene vinylene. These polymers also include homopolymers, copolymers of two or more monomers, and derivatives of these (e.g., substituted compounds with a substituent). Polythiophene includes poly(3,4-ethylenedioxythiophene), for example.

Among the conjugated polymers, a conjugated polymer having a monomer unit corresponding to a pyrrole compound is preferable. When the naphthalene compound D is combined with such a conjugated polymer, a higher bonding strength can be easily achieved. The pyrrole compound should have a pyrrole skeleton. Examples the pyrrole compound include pyrrole, a compound in which an aliphatic ring or a heterocyclic ring is fused to pyrrole, and substituted compound of these (compounds having a substituent). Examples of the substituent include alky groups (including also an aminoalkyl group and a hydroxyalkyl group), amino groups, substituted amino groups, alkoxy groups, hydroxy group, mercapto groups, and halogen atoms. Pyrrole or its condensation compound may have one type of these substituents, or may have two or more types thereof. The conjugated polymer preferably has a repeating structure of monomer units corresponding to a pyrrole compound.

The weight average molecular weight (Mw) of the conjugated polymer is not particularly limited, and is 1000 or more and 1,000,000 or less, for example.

Note that the weight average molecular weight (Mw) in the present description is a value in terms of polystyrene as measured by gel permeation chromatography (GPC). Measurement by GPC usually uses a polystyrene gel column and water/methanol (volume ratio 8/2) as a mobile phase.

The amount of the naphthalene compound D is, for example, 0.1 parts by mass or more and 400 parts by mass or less relative to 100 parts by mass of the conjugated polymer, and may be 1 part by mass or more and 350 parts by mass or less, or may be 10 parts by mass or more and 300 parts by mass or less.

Such an organic conductor, which can suppress an increase in resistance even in high-humidity environments, is highly reliable. As such, the organic conductor can be used for various electronic devices. The organic conductor is particularly suitable for use in solid electrolytes of electrolytic capacitors.

### [Electrolytic Capacitor]

An electrolytic capacitor includes an anode body including a dielectric layer on a surface thereof and a solid electrolyte covering at least a portion of the dielectric layer. The solid electrolyte contains the organic conductor described above. The solid electrolyte constitutes a cathode portion of the electrolytic capacitor.

### (Anode Body)

The anode body can contain a valve metal, an alloy containing the valve metal, and a compound containing the valve metal, for example. These materials can be used singly or in combination of two or more. Examples of the valve metal that can be preferably used include aluminum, tantalum, niobium, and titanium. An anode body with a porous surface can be obtained, for example, by roughening the surface of a substrate (e.g., a foil-like or plate-like substrate) containing a valve metal by etching or the like. The anode body may be a molded body of particles containing a valve metal or a sintered body thereof. Note that the sintered body has a porous structure.

### (Dielectric Layer)

The dielectric layer is formed by anodizing the valve metal on the surface of the anode body by, for example, chemical treatment. The dielectric layer should be formed so as to cover at least a portion of the anode body. The dielectric layer is usually formed on the surface of the anode body. As a result of being formed on the porous surface of the anode body, the dielectric layer is formed along the inner wall surfaces of the holes or dents (pits) of the surface of the anode body.

The dielectric layer contains an oxide of the valve metal. For example, a dielectric layer when tantalum is used as the valve metal contains Ta₂O₅, and a dielectric layer when aluminum is used as the valve metal contains Al₂O₃. Note that the dielectric layer is not limited thereto as long as it functions as a dielectric. When the surface of the anode body is porous, the dielectric layer is formed along the surface (including the inner wall surfaces of the holes) of the anode body.

### (Cathode Portion)

The cathode portion includes at least a solid electrolyte covering at least a portion of the dielectric layer. The cathode portion usually includes a solid electrolyte and a cathode lead out layer covering at least a portion of the solid electrolyte.

### (Solid Electrolyte)

The solid electrolyte contains the organic conductor described above and is formed so as to cover the dielectric layer. The solid electrolyte need not necessarily cover the entirety (entire surface) of the dielectric layer, but should be formed so as to cover at least a portion of the dielectric layer. In an electrolytic capacitor, the solid electrolyte may form a solid electrolyte layer.

The solid electrolyte contains the naphthalene compound D, and may also contain another dopant as necessary. As the other dopant, at least one type selected from the group consisting of an anion and a polyanion is used. Examples of the anion include sulfonate ion, nitrate ion, phosphate ion, borate ion, organic sulfonate ion, and carboxylate ion. Examples of the polyanion include polyvinylsulfonic acid, polystyrenesulfonic acid, polyallylsulfonic acid, polyacrylsulfonic acid, polymethacrylsulfonic acid, polyacrylic acid, and polymethacrylic acid. The polyanion also includes polyester sulfonic acid and phenolic sulfonic acid novolac resin, for example.

From the point of view of more effectively exhibiting the effects of the naphthalene compound D, the ratio of the naphthalene compound D in all dopants is preferably 80 mass % or more, for example, and may be 90 mass % or more or 95 mass % or more. The ratio of the naphthalene compound D in all the dopants is 100 mass % or less. It is possible to use only the naphthalene compound D as a dopant.

That is, the electrolytic capacitor may be an electrolytic capacitor including an anode body including a dielectric layer on the surface thereof and a solid electrolyte covering a part of the dielectric layer, wherein the solid electrolyte contains an organic conductor, the organic conductor contains a conjugated polymer and a dopant, and 80 mass % or more of the dopant is the naphthalene compound D.

The solid electrolyte may contain an additional additive. Examples of the additional additive include known additives other than the dopant and known conductive materials (e.g., conductive inorganic materials such as manganese dioxide) other than the organic conductor.

The solid electrolyte can be formed, for example, by either or both chemical polymerization and electropolymerization of a constituent monomer of a conjugated polymer on the dielectric layer in presence of a high-purity naphthalene compound D. Alternatively, the solid electrolyte covering the dielectric layer can be formed in a manner that a solution in which the conjugated polymer and the naphthalene compound D are dissolved or a dispersion in which the conjugated polymer and the naphthalene compound D are dispersed is brought into contact with the dielectric layer. After the solution or dispersion is brought into contact with the dielectric layer, drying or heating treatment may be performed as necessary.

The solid electrolyte layer may be a single layer or may be composed of a plurality of layers. When the solid electrolyte layer is composed of a plurality of layers, the compositions of the respective layers (e.g., type of the conjugated polymer, type of the dopant or the additive, and the ratio of each component) may be the same as or different from each other.

Between the dielectric layer and the solid electrolyte, a layer or the like to enhance adhesion may be provided as necessary.

### (Cathode Lead Out Layer)

The cathode lead out layer includes, for example, a carbon layer formed on the surface of the solid electrolyte and a metal paste layer formed on the surface of the carbon layer. The cathode layer is formed by sequentially stacking the layers.

The carbon layer can be formed by immersing, in a dispersion containing conductive carbon, the anode body including the dielectric layer at least a portion of which is covered with the solid electrolyte, or by applying a paste containing conductive carbon onto the surface of the solid electrolyte. Graphite such as artificial graphite or natural graphite is used as the conductive carbon, for example. As the dispersion and the paste, conductive carbon dispersed in a water-based liquid medium is used, for example.

The metal paste layer can be formed, for example, by stacking a composition containing metal particles on the surface of the carbon layer. As the metal paste layer, a silver paste layer formed from a composition containing silver particles and a resin (binder resin) can be used, for example. As the resin, a thermoplastic resin may be used, but a thermosetting resin such as an imide-based resin or an epoxy resin is preferably used. The configuration of the cathode layer is not limited thereto, and should be configured to have a current collecting function.

### Others

For example, the electrolytic capacitor is obtained in a manner that a capacitor element including the anode body and the cathode portion are housed in a vessel or sealed with an outer casing or the like. The electrolytic capacitor can be either a chip type or a stack type, or may be a wound type. The configuration of the capacitor element can be selected according to the type of the electrolytic capacitor. The capacitor element may include a cathode body of a metal foil, similar to the anode body, as necessary. When using a metal foil for the cathode body, a separator may be provided between the metal foil and the anode body.

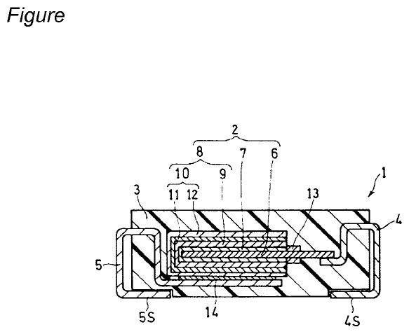

FIG. 1 is a schematic cross-sectional view of the configuration of an electrolytic capacitor according to an embodiment of the present invention. As illustrated in FIG. 1, an electrolytic capacitor 1 includes a capacitor element 2, a resin sealing material 3 that seals the capacitor element 2, and an anode terminal 4 and the cathode terminal 5, with at least portions of both terminals exposed outside the resin sealing material 3. The anode terminal 4 and the cathode terminal 5 may be constituted by a metal such as copper or a copper alloy, for example. The resin sealing material 3 has a substantially rectangular parallelepiped outer shape, and the electrolytic capacitor 1 also has a substantially rectangular parallelepiped outer shape. As the material of the resin sealing material 3, an epoxy resin can be used, for example.

The capacitor element 2 includes an anode body 6, a dielectric layer 7 covering the anode body 6, and a cathode portion 8 covering the dielectric layer 7. The cathode portion 8 includes a solid electrolyte layer 9 covering the dielectric layer 7 and a cathode lead out layer 10 covering the solid electrolyte layer 9. The cathode lead out layer 10 includes a carbon layer 11 and a metal paste layer 12.

The anode body 6 has an area facing the cathode portion 8 and an area not facing the cathode portion 8. An insulative separation layer 13 is formed so as to cover the surface of the anode body 6 in a strip shape in a part adjacent to the cathode portion 8 of the area not facing the cathode portion 8 of the anode body 6, thereby restricting contact between the cathode portion 8 and the anode body 6. Another part of the area not facing the cathode portion 8 is electrically connected to the anode terminal 4 by welding. The cathode terminal 5 is electrically connected to the cathode portion 8 via an adhesive layer 14 formed from a conductive adhesive.

The anode terminal 4 and the cathode terminal 5 respectively have main surfaces 4S and 5S exposed from the same surface of the resin sealing material 3. The exposed surfaces are used, for example, for solder connection to a substrate (not shown) on which the electrolytic capacitor 1 is to be mounted.

### EXAMPLES

Hereinafter, the present invention will be described specifically based on examples and comparative examples. However, the present invention is not limited to the following examples.

The following compounds (A1) to (A5) were used as additives. The naphthalene compound (A1) is 7-sulfo-2-naphthoic acid. The naphthalene compound (A2) is 7-sulfo-1-naphthoic acid. The naphthalene compound (A3) is 6-sulfo-1-naphthoic acid. The compound (A4) is 1-sulfo-2-naphthoic acid. The compound (A5) is 4-sulfo-1-naphthoic acid.

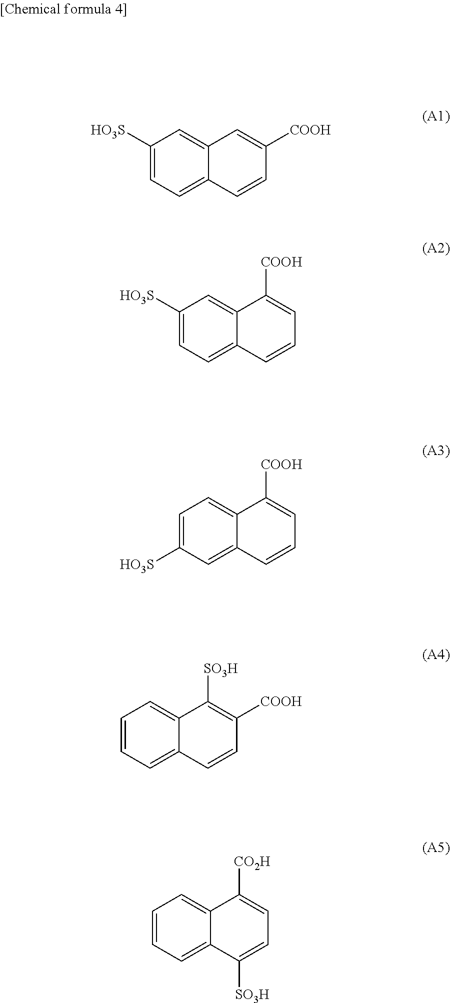 [SMILES: O=C(O)c1ccc2ccc(S(=O)(=O)O)cc2c1 ; O=C(O)c1cccc2ccc(S(=O)(=O)O)cc12 ; O=C(O)c1cccc2cc(S(=O)(=O)O)ccc12 ; O=C(O)c1ccc2ccccc2c1S(=O)(=O)O ; O=C(O)c1ccc(S(=O)(=O)O)c2ccccc12]

### Examples 1 to 5

### (Electrolytic Capacitor Production)

In the following manner, an electrolytic capacitor 1 illustrated in FIG. 1 was produced to evaluate the characteristics thereof.

### (1) Step of Preparing Anode Body 6

The surface of an aluminum foil (thickness: 100 μm) as a substrate was roughened by etching to prepare an anode body 6.

### (2) Step of Forming Dielectric Layer 3

The anode body 6 was immersed in a phosphoric acid solution with a concentration of 0.3 mass % at a temperature of 70° C., and subjected to a DC voltage of 70 V for 20 minutes to form a dielectric layer 7 containing aluminum oxide.

### (3) Step of Forming Solid Electrolyte Layer 9

A solid electrolyte layer 9 containing polypyrrole and any of the naphthalene compounds D (A1) to (A5) (purity of 99% or more) each as a dopant was formed on the dielectric layer 7 by electrolytic polymerization in the following manner.

First, an aqueous solution containing a pyrrole monomer and any of the additives (A1) to (A5) was prepared. The concentration of the pyrrole monomer in the aqueous solution was set to 0.5 mol/L, and the concentration of the naphthalene compound D was set to 0.3 mol/L. Sulfuric acid was added to the aqueous solution to adjust pH to 3.0

The anode body 2 on which the dielectric layer has been formed in (2) described above and a counter electrode were immersed in the resulting aqueous solution, followed by electrolytic polymerization at a polymerization voltage of 3V and at 25° C. to form a solid electrolyte layer 9.

### (4) Step of Forming Cathode Lead Out Layer 10

A dispersion of graphite particles dispersed in water was applied onto the surface of the solid electrolyte layer 9 obtained in (3) described above, and dried in air to form a carbon layer 11. Next, a silver paste containing silver particles and an epoxy resin was applied onto the surface of the carbon layer 11, followed by heating to form a metal paste layer 12. Thus, a cathode lead out layer 10 composed of a carbon layer 11 and a metal paste layer 12 was formed.

In the manner described above, a capacitor element 2 was produced.

### (5) Electrolytic Capacitor Assembly

One end of a cathode terminal 5 and the cathode lead out layer 10 of the capacitor element 2 obtained in (4) described above were bonded with a conductive adhesive 14. One end of the anode body 6 protruding from the capacitor element 2 and one end of an anode terminal 4 were bonded by laser welding.

Next, a resin sealing material 3 formed from an insulative resin was formed around the capacitor element 2. In the formation, the other end of the anode terminal 4 and the other end of the cathode terminal 5 were arranged to be pulled out from the resin sealing material 3.

In the manner described above, electrolytic capacitors 1 with a rated voltage of 2 V and a static capacitance of 30 μF were produced.

### (6) Evaluation

The following evaluations were carried out for each of the electrolytic capacitors and each of the additives.

### (a) ΔESR

The initial ESR (=Z₀) (mΩ) of the electrolytic capacitor at a frequency of 100 kHz was measured in a 20° C. environment using an LCR meter for four-terminal measurement.

After applying the rated voltage to the electrolytic capacitor for 125 hours in an environment at 85° C. and 85% RH, the ESR (=Z) (mΩ) was measured in a 20° C. environment in the same manner as that for measuring the initial ESR. A ΔESR was determined using ΔESR=(Z−Z0)/Z0. Here, the ΔESR was a rate of change in a high-humidity environment.

### (b) Interaction Energy

The interaction energy between the additive and polypyrrole was calculated by the procedure described above.

### Comparative Example 1

An electrolytic capacitor was produced and evaluated in the same manner as in Example 1 except that naphthalene sulfonic acid was used in place of the naphthalene compound (A1).

### Comparative Example 2

An electrolytic capacitor was produced and evaluated in the same manner as in Example 1 except that 6-sulfo-2-naphthoic acid was used in place of the naphthalene compound (A1).

### Comparative Example 3

An electrolytic capacitor was produced and evaluated in the same manner as in Example 1 except that 5-sulfo-1-naphthoic acid was used in place of the naphthalene compound (A1).

### Comparative Example 4

The 2Na sulfo-1-naphthoate of Example 1 in Patent Literature 2 is a mixture containing the dopant compounds shown below in a ratio of 5:10:40:10:35 in order from the left.

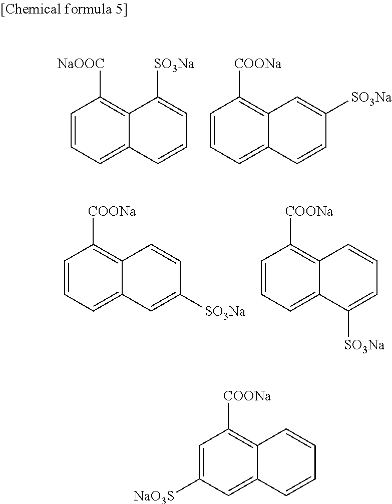

An electrolytic capacitor was produced and evaluated in the same manner as in Example 1 except that the above mixture of 2Na sulfo-1-naphthoate was used in place of the naphthalene compound (A1).

### Comparative Example 5

The 2Na sulfo-2-naphthoate of Example 2 in Patent Literature 2 is a mixture containing the dopant compounds shown below in a ratio of 20:35:5:35:5 in order from the left.

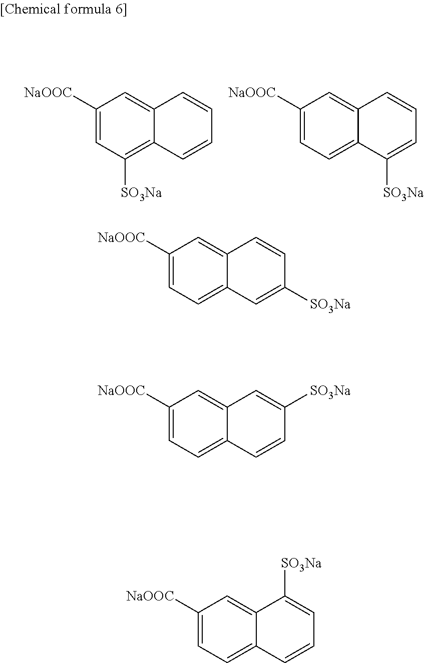

An electrolytic capacitor was produced and evaluated in the same manner as in Example 1 except that the above mixture of 2Na sulfo-2-naphthoate was used in place of the naphthalene compound (A1).

### Comparative Example 6

The sulfo-1-naphthoic acid of Example 4 in Patent Literature 2 is a mixture containing the dopant compounds shown below in a ratio of 5:10:40:10:35 in order from the left.

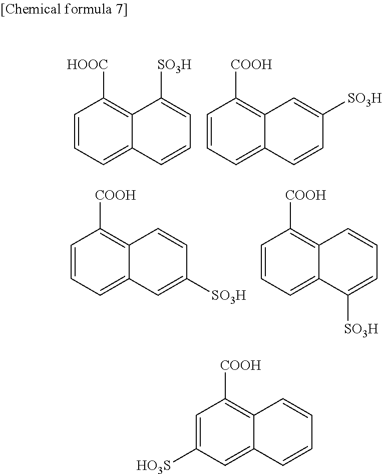

An electrolytic capacitor was produced and evaluated in the same manner as in Example 1 except that the above mixture of sulfo-1-naphthoic acid was used in place of the naphthalene compound (A1).

### Comparative Example 7

The sulfo-2-naphthoic acid of Example 5 in Patent Literature 2 is a mixture containing the dopant compounds shown below in a ratio of 20:35:5:35:5 in order from the left.

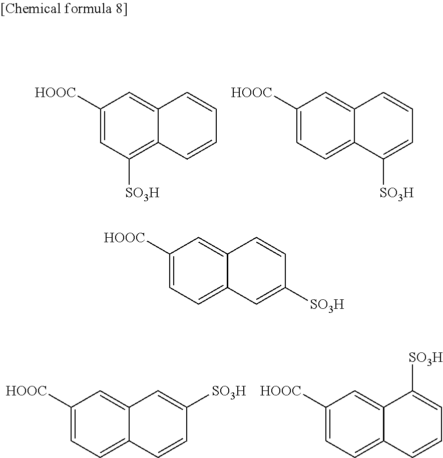

An electrolytic capacitor was produced and evaluated in the same manner as in Example 1 except that the above mixture of sulfo-2-naphthoic acid was used in place of the naphthalene compound (A1).

Table 1 shows the evaluation results of Examples and Comparative Examples. In Table 1, E1 to E5 refer to Examples 1 to 5, respectively, and C1 to C7 refer to Comparative Examples 1 to 7, respectively.

TABLE 1

| | Additive | | |
|---|---|---|---|
| | | Interaction energy | |
| | Type | (kcal/mol) | Δ ESR |
| E1 | 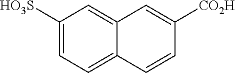 [SMILES: O=C(O)c1ccc2ccc(S(=O)(=O)O)cc2c1] | −20.90 | 0.010 |
| E2 | 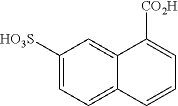 [SMILES: O=C(O)c1cccc2ccc(S(=O)(=O)O)cc12] | −20.79 | 0.010 |
| E3 | 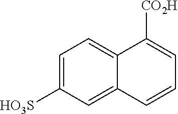 [SMILES: O=C(O)c1cccc2cc(S(=O)(=O)O)ccc12] | −17.46 | 0.072 |
| E4 | 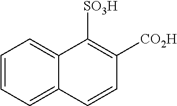 [SMILES: O=C(O)c1ccc2ccccc2c1S(=O)(=O)O] | −13.86 | 0.78 |
| E5 | 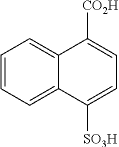 [SMILES: O=C(O)c1ccc(S(=O)(=O)O)c2ccccc12] | −13.63 | 0.90 |
| C1 | Naphthalene sulfonic acid | about −10 | 8.8 |
| C2 | 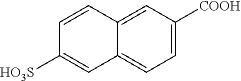 [SMILES: O=C(O)c1ccc2cc(S(=O)(=O)O)ccc2c1] | −11.78 | 2.9 |
| C3 | 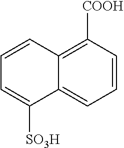 [SMILES: O=C(O)c1cccc2c(S(=O)(=O)O)cccc12] | −12.40 | 1.9 |
| C4 | 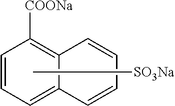 | | 0.019 |
| C5 | 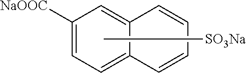 | | 0.014 |
| C6 | 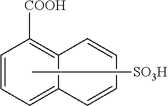 | | 0.015 |
| C7 | 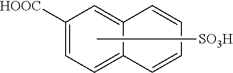 | | 0.015 |

As shown in Table 1, in E1 to E5 respectively using the additive A1 to A5, an increase in rate of change of ESR in high-humidity environments was particularly reduced as compared with C1 to C3 using the naphthalene sulfonic acids. The ΔESR became smaller as the interaction energy between the additive and the conjugated polymer decreased. From this fact, it is thought that a high bonding strength between the naphthalene compound D and the conjugated polymer was obtained in each of E1 to E5, with a result that suppression of the increase in resistance of the solid electrolyte layer and the increase in ESR was achieved even in high-humidity environments.

### ADDITIONAL REMARKS

According to the description of the embodiments provided above, the following techniques are disclosed.

### Technique 1

An additive for an organic conductor, including a naphthalene compound having a naphthalene ring, a sulfo group bonded to the naphthalene ring, and a carboxy group bonded to the naphthalene ring, wherein
a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded, and
the naphthalene compound has a purity of 41 mass % or more.

### Technique 2

The additive for an organic conductor according to Technique 1, wherein the naphthalene compound is at least one selected from the group consisting of a 7-sulfo-2-naphthoic acid compound, a 7-sulfo-1-naphthoic acid compound, a 6-sulfo-1-naphthoic acid compound, a 1-sulfo-2-naphthoic acid compound, and a 4-sulfo-1-naphthoic acid compound.

### Technique 3

The additive for an organic conductor according to Technique 1 or 2, wherein the additive has an interaction energy to a conjugated polymer of −13 kcal/mol or less.

### Technique 4

The additive for an organic conductor according to any one of Techniques 1 to 3, wherein the naphthalene compound has a content ratio of 80 mass % or more.

### Technique 5

An organic conductor including a conjugated polymer and the additive according to any one of Techniques 1 to 4.

### Technique 6

The organic conductor according to Technique 5, wherein the conjugated polymer has a monomer unit corresponding to a pyrrole compound.

### Technique 7

An electrolytic capacitor including: an anode body including a dielectric layer on a surface thereof, and a solid electrolyte covering a portion of the dielectric layer, wherein the solid electrolyte contains the organic conductor according to Technique 5 or 6.

### Technique 8

An electrolytic capacitor including: an anode body including a dielectric layer on a surface thereof; and a solid electrolyte covering a portion of the dielectric layer, wherein
the solid electrolyte contains an organic conductor,
the organic conductor contains a conjugated polymer and a dopant,
a naphthalene compound constitutes 80% or more of the dopant,
the naphthalene compound has a naphthalene ring, a sulfo group bonded to the naphthalene ring, and a carboxy group bonded to the naphthalene ring,
a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded.

### Technique 9

An electrolytic capacitor including the organic conductor according to Technique 8, wherein the naphthalene compound has a purity of 41 mass % or more.

### Technique 10

An organic conductor including: a conjugated polymer; and a dopant, wherein
the dopant contains a naphthalene compound,
the naphthalene compound has a naphthalene ring, a sulfo group bonded to the naphthalene ring, and a carboxy group bonded to the naphthalene ring,
   a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded, and
the naphthalene compound has a purity of 41 mass % or more.

### Technique 11

An additive for an organic conductor, wherein a content ratio of at least one selected from the group consisting of a 7-sulfo-2-naphthoic acid compound, a 7-sulfo-1-naphthoic acid compound, and a 6-sulfo-1-naphthoic acid compound is 80 mass % or more.

### Technique 12

The additive for an organic conductor according to Technique 11, wherein a content ratio of at least one selected from the group consisting of 7-sulfo-2-naphthoic acid and a 7-sulfo-1-naphthoic acid compound is 80 mass % or more.

### Technique 13

An organic conductor including: a conjugated polymer; and a dopant, wherein
80 mass % or more of the dopant is at least one selected from the group consisting of a 7-sulfo-2-naphthoic acid compound, a 7-sulfo-1-naphthoic acid compound, and a 6-sulfo-1-naphthoic acid compound.

### Technique 14

The organic conductor according to Technique 13, wherein
80 mass % or more of the dopant is at least one selected from the group consisting of 7-sulfo-2-naphthoic acid and a 7-sulfo-1-naphthoic acid compound.

Although the present invention has been described in terms of the presently preferred embodiments, it is to be understood that such disclosure is not to be interpreted as limiting. Various alterations and modifications will no doubt become apparent to those skilled in the art to which the present invention pertains, after having read the above disclosure. Accordingly, it is intended that the appended claims be interpreted to cover all alterations and modifications as falling within the true spirit and scope of the invention.

### INDUSTRIAL APPLICABILITY

According to the present disclosure, it is possible to provide an additive that can significantly increase moisture resistance of an organic conductor. Such an additive can be used in organic conductors for various electronic devices such as electrolytic capacitors to stabilize product quality even in high-humidity environments.

### EXPLANATION OF SIGNS

1: electrolytic capacitor, 2: capacitor element, 3: resin sealing material, 4: anode terminal, 4S: main surface of anode terminal, 5: cathode terminal, 5S: main surface of cathode terminal, 6: anode body, 7: dielectric layer, 8: cathode portion, 9: solid electrolyte layer, 10: cathode lead out layer, 11: carbon layer, 12: metal paste layer, 13: separation layer, 14: adhesive layer

1. An additive for an organic conductor, comprising a naphthalene compound having a naphthalene ring, a sulfo group bonded to the naphthalene ring, and a carboxy group bonded to the naphthalene ring, wherein
a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded, and
the naphthalene compound has a purity of 41 mass % or more.

2. The additive for an organic conductor according to claim 1, wherein
the naphthalene compound is at least one selected from the group consisting of a 7-sulfo-2-naphthoic acid compound, a 7-sulfo-1-naphthoic acid compound, a 6-sulfo-1-naphthoic acid compound, a 1-sulfo-2-naphthoic acid compound, and a 4-sulfo-1-naphthoic acid compound.

3. The additive for an organic conductor according to claim 1, wherein
the additive has an interaction energy to a conjugated polymer of −13 kcal/mol or less.

4. The additive for an organic conductor according to claim 1, wherein
the naphthalene compound has a content ratio of 80 mass % or more.

5. An organic conductor comprising a conjugated polymer and the additive according to claim 1.

6. The organic conductor according to claim 5, wherein
the conjugated polymer has a monomer unit corresponding to a pyrrole compound.

7. An electrolytic capacitor comprising:
an anode body including a dielectric layer on a surface thereof; and
a solid electrolyte covering a portion of the dielectric layer, wherein
the solid electrolyte contains the organic conductor according to claim 5.

8. An electrolytic capacitor comprising:
an anode body including a dielectric layer on a surface thereof; and
a solid electrolyte covering a portion of the dielectric layer, wherein
the solid electrolyte contains an organic conductor,
the organic conductor contains a conjugated polymer and a dopant,
a naphthalene compound constitutes 80 mass % or more of the dopant,
the naphthalene compound has a naphthalene ring, a sulfo group bonded to the naphthalene ring, and a carboxy group bonded to the naphthalene ring,
a first carbon atom and a second carbon atom are adjacent to each other, or a number n of carbon atoms between the first carbon atom and the second carbon atom is 3 or less, the first carbon atom being a carbon atom of the naphthalene ring to which the sulfo group is bonded, the second carbon atom being a carbon atom of the naphthalene ring to which the carboxy group is bonded.

9. An electrolytic capacitor comprising the organic conductor according to claim 8, wherein
the naphthalene compound has a purity of 41 mass % or more.

10. An additive for an organic conductor, wherein
a content ratio of at least one selected from the group consisting of a 7-sulfo-2-naphthoic acid compound, a 7-sulfo-1-naphthoic acid compound, and a 6-sulfo-1-naphthoic acid compound is 80 mass % or more.

11. The additive for an organic conductor according to claim 10, wherein
a content ratio of at least one selected from the group consisting of 7-sulfo-2-naphthoic acid and a 7-sulfo-1-naphthoic acid compound is 80 mass % or more.

12. An organic conductor comprising:
a conjugated polymer; and
a dopant, wherein
80 mass % or more of the dopant is at least one selected from the group consisting of a 7-sulfo-2-naphthoic acid compound, a 7-sulfo-1-naphthoic acid compound, and a 6-sulfo-1-naphthoic acid compound.

13. The organic conductor according to claim 12, wherein
80 mass % or more of the dopant is at least one selected from the group consisting of 7-sulfo-2-naphthoic acid and a 7-sulfo-1-naphthoic acid compound.
